(19) United States
(12) Patent Application Publication
(10) Pub. No.: US 2008/0299176 A1
(43) Pub. Date: Dec. 4, 2008

(54) DRUG DELIVERY DEVICE COMPRISING CROSSLINKED POLYURETHANE-SILOXANE-CONTAINING COPOLYMERS

(76) Inventors: Yu-Chin Lai, Pittsford, NY (US); Ruiwen Shi, Webster, NY (US); O. William Lever, JR., Pittsford, NY (US); Dominic V. Ruscio, Webster, NY (US); Yan Huang, Webster, NY (US)

Correspondence Address: Bausch & Lomb Incorporated, One Bausch & Lomb Place, Rochester, NY, 14604-2701, US

(21) Appl. No.: 11/755,211

(22) Filed: May 30, 2007

(51) Int. Cl. A61F 2/14; A61K 31/335; A61K 31/337; A61K 31/433; A61K 31/56; A61K 31/58; A61K 36/06; A61K 47/48; A61P 27/00

(52) U.S. Cl. 424/427; 424/195.15; 514/169; 514/172; 514/178; 514/363; 514/449; 514/450; 514/772.3

(57) ABSTRACT

A drug delivery device for placement in the eye includes a drug core comprising a hydrophobic pharmaceutically active agent, and a holder that holds the drug core. The holder is made of a material impermeable to passage of the active agent and includes an opening for passage of the pharmaceutically agent therethrough to eye tissue. The device includes polyurethane-siloxane-containing copolymers crosslinked with hydrophilic monomers.

### CROSS REFERENCE

This application claims the benefit of Provisional Patent Application No. 60/638,480 filed Dec. 22, 2004 and is incorporated herein by reference.

### FIELD OF THE INVENTION

This invention relates to a drug delivery device, preferably a device that is placed or implanted in the eye to release a pharmaceutically active agent to the eye. The device includes a drug core and a holder for the drug core, wherein the holder is made of a material impermeable to passage of the active agent and includes at least one opening for passage of the pharmaceutically agent therethrough to eye tissue. Particularly, this invention provides improved methods of making such devices by using polyurethane-siloxane-containing copolymers.

### BACKGROUND OF THE INVENTION

In the field of drug delivery, it is important to be able to control the release profile of the therapeutic agent. Release profiles can include an initial large burst effect, followed by an exponential decrease in the rate of drugs released, an initial large burst effect followed by a decrease to an essentially constant release rate over time, and essentially constant rate (zero-order) kinetics release behavior, sometimes with an initial non-therapeutically significant moderate burst effect.

Because the successful treatment of a patient with a disease state can depend upon the amount and timing of delivery of an active agent, there is still a need for new materials that can serve as a release rate modifying barrier, especially in implantable drug delivery devices. We have discovered that a polyurethane-siloxane block copolymer can serve that function for relatively hydrophobic drugs such as fluocinolone acetonide (FA). Disclosed herein is the use of such a copolymer as a barrier film in an intraocular drug delivery device.

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye and treating various eye diseases. Examples are found in the following patents, the disclosures of which are incorporated herein by reference: US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Drug Delivery Device, filed Mar. 28, 2003) (Mosack et al.); and U.S. patent application Ser. No. 10/610,063 (Drug Delivery Device, filed Jun. 30, 2003) (Mosack).

Many of these devices include an inner drug core including a pharmaceutically active agent, and some type of holder for the drug core made of an impermeable material such as silicone or other hydrophobic materials. The holder includes one or more openings for passage of the pharmaceutically agent through the impermeable material to eye tissue. Many of these devices include at least one layer of material permeable to the active agent, such as polyvinyl alcohol. Although entirely suitable in some drug release applications, there is still a need for new formulations that allow for a tailored release profile dependant upon the properties of the active in the drug core, such as its degree of hydrophobic or hydrophilic character.

This invention provides a drug delivery device comprising a holder made of a material impermeable to passage of a pharmaceutically active agent, and including at least one opening for passage of the active agent therethrough, a drug core contained in the holder, and including a hydrophobic pharmaceutically active agent and a disc made of a material permeable to passage of the active agent, the disc contained in the holder and disposed between the drug core and the at least one opening in the holder, the disk comprising crosslinked polyurethane-siloxane-containing copolymers.

The polyurethane-siloxane copolymer of this invention is prepared by reacting a polyurethane-siloxane-prepolymer with at least two ethylenically unsaturated groups and a hydrophilic monomer crosslinking agent such as dimethyl acrylamide.

### BRIEF DESCRIPTION OF THE DRAWINGS

FIG. 1 is a perspective view of a first embodiment of a drug delivery device of this invention.

FIG. 2 is a cross-sectional view of the device of FIG. 1.

FIG. 3 is a cross-sectional view of the device of FIGS. 1 and 2 during assembly.

FIG. 4 is a cross-sectional view of a second embodiment of a drug delivery device.

### DETAILED DESCRIPTION OF PREFERRED EMBODIMENTS

The term “prepolymer” denotes a high molecular weight monomer containing polymerizable groups. The monomers added to the monomeric mixture of the present invention may therefore be low molecular weight monomers or prepolymers.

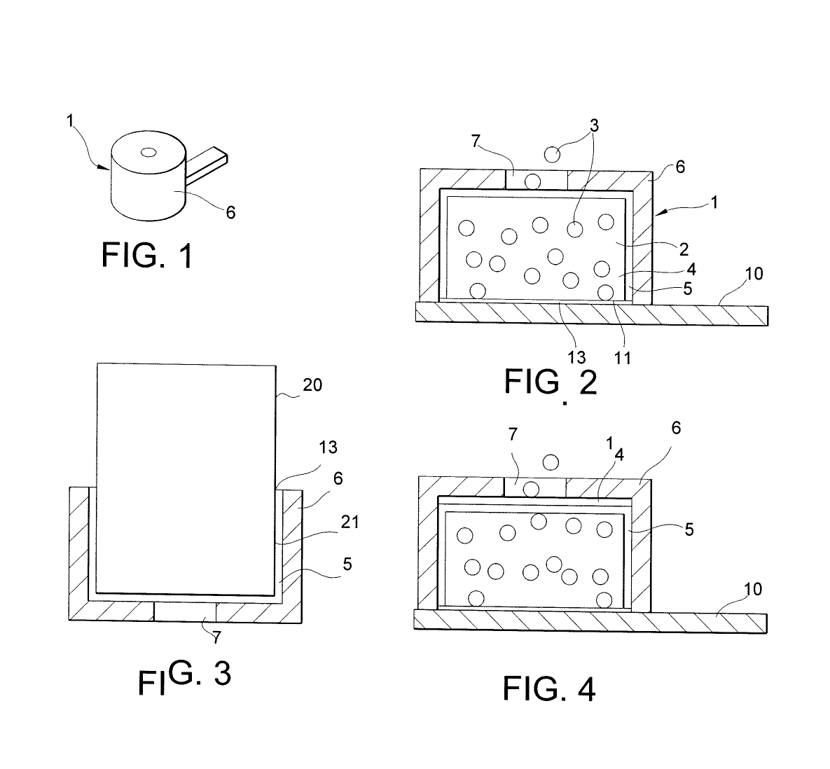

FIGS. 1 and 2 illustrate a first embodiment of a device of this invention. Device 1 is a sustained release drug delivery device for implanting in the eye. Device 1 includes inner drug core 2 including a pharmaceutically active agent 3.

This active agent may include any compound, composition of matter, or mixture thereof that can be delivered from the device to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; benzodiazepine receptor agonists such as abecarnil; GABA receptor modulators such as baclofen, muscimol and benzodiazepines; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrase inhibitors; diagnostic agents; antiapoptosis agents; gene therapy agents; sequestering agents; reductants such as glutathione; antipermeability agents; antisense compounds; antiproliferative agents; antibody conjugates; antidepressants; blood flow enhancers; antiasthmatic drugs; antiparasiticagents; non-steroidal anti inflammatory agents such as ibuprofen; nutrients and vitamins: enzyme inhibitors: antioxidants; anticataract drugs; aldose reductase inhibitors; cytoprotectants; cytokines, cytokine inhibitors and cytokine protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone and triamcinolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; svmpathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

For this invention, relatively hydrophobic drugs such as fluocinolone acetonide, triamcinolone acetonide, loteprednol etabonate, dexamethasone, acetazolamide, indomethacin, amphotericin B, paclitaxel, abecarnil, cyclosporines, etc. are preferred for use with the crosslinked polyurethane-siloxane copolymers.

As shown in the illustrated embodiment, active agent 3 may be mixed with a matrix material 4. Preferably, matrix 4 is made of pharmaceutically acceptable materials that are compatible with body fluids and the eye, these include, but not limit to, mannitol, dicalcium phosphate, calcium sulfate, lactose, talc, stearic acid, aluminum stearate, magnesium stearate, colloidal silicon dioxide, clays, cellulose, kaolin, starch, microcrystalline cellulose, gelatin, sodium alginate, methylcellulose, ethylcellulose, carboxymethylcellulose, croscarmelose, crospovidone, hydroxypropylcellulose, hydroxypropylmethylcellulose, polyvinylpyrrolidone (PVP), Veegum, polyethylene glycol, poly(vinyl alcohol) (PVA). Additionally, the matrix should be permeable to passage of the active agent 3 therethrough, particularly when the device is exposed to body fluids. For the illustrated embodiment, the matrix contains PVA as one of its components. However, the matrix material can also be the polyurethane-siloxane-containing copolymers of coating 5. Also, in this embodiment, inner drug core 2 may be coated with a coating 5 of additional matrix material which may be the same or different from material 4 mixed with the active agent. For the illustrated embodiment, the coating employed is polyurethane-siloxane-containing copolymer.

Device 1 includes a holder 6 for the inner drug core 2. Holder 6 is made of a material that is impermeable to passage of the active agent 3 therethrough. Since holder 6 is made of the impermeable material, at least one passageway 7 is formed in holder 6 to permit active agent 3 to pass therethrough and contact eye tissue. In other words, active agent passes through any permeable matrix material 4 and permeable coating 5, and exits the device through passageway 7. For the illustrated embodiment, the holder is made of silicone, especially polydimethylsiloxane (PDMS) material.

A method of making a device of the type shown in FIGS. 1 and 2 includes the following procedures. A cylindrical cup of silicone is separately formed, for example by molding, having a size generally corresponding to the drug core tablet and a shape as generally shown in FIG. 2. This silicone holder is then extracted with a solvent such as isopropanol. Openings 7 are placed in silicone, for example, by boring or with the laser. A drop of liquid PVA is placed into the holder through the open end 13 of the holder, this open end best seen in FIG. 3. Then, the inner drug core tablet is placed into the silicone holder through the same open end 13 and pressed into the cylindrical holder. In this method, as a result, the pressing of the tablet causes the liquid PVA to fill the space between the tablet inner core and the silicone holder, thus forming permeable layer 5 shown in FIGS. 1 and 2. For the illustrated embodiment, a layer of adhesive 11 is applied to the open end 13 of the holder to fully enclose the inner drug core tablet at this end. Tab 10 is inserted at this end of the device. The liquid PVA and adhesive are cured by heating the assembly.

For the illustrated embodiment, the active agent may be provided in the form of a micronized powder, and then mixed with an aqueous solution of the matrix material, in this case crosslinked polyurethane-siloxane-containing copolymer, whereby the active agent and polyurethane-siloxane-containing copolymer agglomerate into larger sized particles. The resulting mixture is then dried to remove some of the moisture, and then milled and sieved to reduce the particle size so that the mixture is more flowable. Optionally, a small amount of inert lubricant, for example, magnesium stearate, may be added to assist in tablet making. This mixture is then formed into a tablet using standard tablet making apparatus, this tablet representing inner drug core 2.

An alternate embodiment is illustrated in FIG. 4. In this embodiment, the device further includes a disc 14 made of permeable material covering passageway 7 between the holder 6 and layer 5. For the illustrated embodiment, disc 14 may be preformed from polyurethane-siloxane-containing copolymer, similar to the material used for layer 5 and matrix material 4. In assembling this embodiment, disc 14 is placed in holder 6 prior to adding the liquid curable material forming layer 5. Then, pin 20 is used to displace the liquid, as in the previous embodiment. A potential advantage of this embodiment is that the thickness of the permeable materials at passageway 7 can be controlled better, thereby providing more consistent release of active through the permeable materials into passageway 7.

In addition to the illustrated materials, a wide variety of materials may be used to construct the devices of the present invention. The only requirements are that they are inert; non-immunogenic and of the desired permeability. Materials that may be suitable for fabricating the device include naturally occurring or synthetic materials that are biologically compatible with body fluids and body tissues, and essentially insoluble in the body fluids with which the material will come in contact. The use of rapidly dissolving materials or materials highly soluble in body fluids are to be avoided since dissolution of the wall would affect the constancy of the drug release, as well as the capability of the device to remain in place for a prolonged period of time.

Naturally occurring or synthetic materials that are biologically compatible with body fluids and eye tissues and essentially insoluble in body fluids which the material will come in contact include, but are not limited to, glass, metal, ceramics, polyurethane-polysiloxane-containing copolymers, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

Hydrophilic monomers may be combined with the polyurethane-siloxane prepolymer to serve as crosslinking agents. Examples of hydrophilic monomers include, but are not limited to, ethylenically unsaturated lactam-containing monomers such as N-vinyl pyrrolidinone; methacrylic and acrylic acids; (meth)acrylic substituted alcohols, such as 2-hydroxyethylmethacrylate (HEMA) and 2-hydroxyethylacrylate; and (meth)acrylamides, such as methacrylamide and N,N-dimethyl acrylamide (DMA); vinyl carbonate or vinyl carbamate monomers such as disclosed in U.S. Pat. Nos. 5,070,215; and oxazolinone monomers such as disclosed in U.S. Pat. No. 4,910,277. Other hydrophilic monomers such as glycerol methacrylate and polyethyleneglycol monomethacrylate are also useful in the present invention.

Preferred hydrophilic monomers which may be incorporated into the hydrogel of the present invention include hydrophilic monomers such as N,N-dimethyl acrylamide (DMA), 2-hydroxyethyl methacrylate, glycerol methacrylate, 2-hydroxyethyl methacrylamide, methacrylic acid and acrylic acid, with DMA being the most preferred. Other suitable hydrophilic monomers will be apparent to one skilled in the art. When the hydrophilic monomer is present as a comonomer, the relative weight % of hydrophilic monomer(s) to total weight % of the comonomer mix is preferably from about 0.5% to 80%, more preferably from about 1% to 75%, even more preferably 10% to 30%. The drug delivery copolymer may also contain ethylenically unsaturated groups as crosslinking agents. In that case, additional crosslinking agents which may be incorporated into the polyurethane-siloxane-containing hydrogel of the present invention include polyvinyl, typically di- or tri-vinyl monomers, most commonly the di- or tri(meth)acrylates of dihydric ethylene glycol, triethylene glycol, butylene glycol, hexane-1,6-diol, thio-diethylene glycol-diacrylate and methacrylate; neopentyl glycol diacrylate; trimethylolpropane triacrylate and the like. The drug delivery film or coating of the invention herein may also contain vinyl and methacrylate containing endgroups such as HEMA.

The illustrated embodiment includes a tab 10 which may be made of a wide variety of materials, including those mentioned above for the matrix material and/or the holder. Tab 10 may be provided in order to attach the device to a desired location in the eye, for example, by suturing. For the illustrated embodiment, tab 10 is made of PVA and is adhered to the inner drug core 2 with adhesive 11. Adhesive 11 may be a curable silicone adhesive, a curable PVA solution, or the like. If it is not necessary to suture the device in the eye, element 10 may have a smaller size such that it does not extend substantially beyond holder 6.

According to preferred embodiments, the holder is extracted to remove residual materials therefrom. For example, in the case of silicone, the holder may include lower molecular weight materials such as unreacted monomeric material and oligomers. It is believed that the presence of such residual materials may also deleteriously affect adherence of the holder surfaces. The holder may be extracted by placing the holder in an extraction solvent, optionally with agitation. Representative solvents are polar solvents such as isopropanol, heptane, hexane, toluene, tetrahydrofuran (THF), chloroform, supercritical carbon dioxide, and the like, including mixtures thereof. After extraction, the solvent is preferably removed from the holder, such as by evaporation in a nitrogen box, a laminar flow hood or a vacuum oven.

If desired, the holder may be plasma treated, following extraction, in order to increase the wettability of the holder and improve adherence of the drug core and/or the tab to the holder. Such plasma treatment employs oxidation plasma in an atmosphere composed of an oxidizing media such as oxygen or nitrogen containing compounds: ammonia, an aminoalkane, air, water, peroxide, oxygen gas, methanol, acetone, alkylamines, and the like or appropriate mixtures thereof including inert gases such as argon. Examples of mixed media include oxygen/argon or hydrogen/methanol. Typically, the plasma treatment is conducted in a closed chamber at an electric discharge frequency of 13.56 MHz, preferably between about 20 to 500 watts at a pressure of about 0.1 to 1.0 torr, preferably for about 10 seconds to about 10 minutes or more, more preferably about 1 to 10 minutes.

The device may be sterilized and packaged. For example, the device may be sterilized by irradiation with gamma radiation.

It will be appreciated the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease states, type of mammalian organism, location of administration, and agents or agent administered are among the factors which would affect the desired size of the sustained release drug delivery device. However, because the device is intended for placement in the eye, the device is relatively small in size. Generally, it is preferred that the device, excluding the suture tab, has a maximum height, width and length each no greater than 10 mm, more preferably no greater than 5 mm, and most preferably no greater than 3 mm.

### EXAMPLES

### Example 1

### Preparation of α,ω-bis(4-hydroxybutyl)polydimethylsiloxane of Mn 4000

A 2-L, three-neck round bottom flask equipped with a reflux condenser, was charged with 50.8 grams (0.182 moles) of 1,3-bishydroxybutyl tetraethyldisiloxane, 985.6 grams (8.1 moles) of dimethoxydimethylsilane, 145.8 grams (8.1 moles) of distilled water and 18.2 mL of concentrated hydrochloric acid. The mixture was heated at 60° C. for 1 hour. Methanol was then distilled off over a 5 hour period, with 650 mL collected. Six hundred fifty mL of 6N hydrochloric acid was then added and the contents were refluxed for 4 hours. The crude product was then separated from the aqueous layer. Ether was added and the solution was extracted with 0.5 N sodium bicarbonate solution twice and then with distilled water until the wash was neutral. The product was then added slowly into an equal weight of a mixture of methanol/water (77.5/22.5). The bottom organic layer was separated, added with ether and dried with magnesium sulfate. Ether was then stripped under vacuum at room temperature and the residue was further stripped under vacuum (0.05 mm torr) at 80° C. to give the final product (510 grams). The molecular weight (Mn) as determined by titration was 4044.

### Example 2

### Preparation of a polymethylsiloxane-based polyurethane copolymer (13D2S4H)

A dry 3-neck, 1000 mL round bottom flask was connected to a nitrogen inlet tube and a reflux condenser linked. Then, isophorone diisocyanate (16.91 g, 0.0761 mole), diethylene glycol (4.038 g, 0.0380 mole), dibutyl tin dilaurate (0.383 g) and 140 mL of methylene chloride were added into the flask all at once and the contents were refluxed. After 16 hours, the amount of isocyanate was determined to decrease to 47.0% by titration. Then α,ω-bis(4-hydroxybutyl)polydimethylsiloxane (102.56 g, 0.02536 mole) from Example 1, was added into the flask. The refluxing was continued for 33 hours, and the amount of isocyanate was dropped down to 14.1% of the original by titration. The contents were then cooled down to ambient temperature. 2-hydroxyethyl methacrylate (2.2928 g) and 1,1′-bi-2-phenol (0.0129 g) were then added and the contents were stirred at ambient temperature until the isocyanate peak at 2267 cm⁻¹ disappeared from the IR spectrum of the product (about 20 hours). The solvent was then stripped under reduced pressure to give product in quantitative yield.

### Examples 3-5

### Preparation of Films from Prepolymer of Example 2

The following monomer mixes were prepared (all weights in parts) and then placed between two silane-treated glass plates. They were then cured under UV (4000 microwatts) for one hour. The cured films were released from plate and extracted with isopropanol overnight. They were then placed in distilled water. Water content of these films was measured gravimetrically.

| | Example | | |
|---|---|---|---|
| Formulation | 3 | 4 | 5 |
| I3D2S4H | 100 | 90 | 70 |
| DMA | 0 | 10 | 30 |
| n-hexanol | 30 | 30 | 30 |
| Darocur-1173 | 0.3 | 0.3 | 0.3 |
| % water | 1.7 | 12 | 39 |

### Example 6

### Diffusion of FA through the Copolymer Membranes

The diffusion was carried out using Valia-Chien diffusion apparatus. FA and membranes with a composition described in Example 5 were used. FA (2 mg) and PBS (3.2 mL) were added to each of the donor cells, and 3.2 mL PBS were added in each of the receptor cells. The cells were capped and the temperature was kept at 37° C. using a circulating water bath. At given time intervals, the diffusion media in the receptor cells were removed and replaced by fresh PBS. FA concentration in the release media was determined using a set of HP series 1100 HPLC instruments equipped with a degasser, a binary pump, an auto sampler and a DAD detector. The column used was an Altima C18 (5μ, 250 mm×4.6 mm). The mobile phase was 40% ACN in water and the flow rate was 1.0 mL/m in. The eluted FA was detected at 238 nm. The concentration of FA in the media was directly determined using HPLC. The diffusion rate of FA through the copolymer membranes with a thickness of 100 μm was estimated to be 1.4 μg/cm²/hour.

### Example 7

Fifty microliters of the monomer mixture of example 5 is placed into a silicone cylindrical holder through the open end of the holder. Then, the inner drug core tablet is placed and pressed into the holder through the same open end. The pressing of the tablet leads to the monomer mixture to fill the space between the tablet inner core and the silicone holder. A layer of silicone adhesive is applied to the open end of the holder to fully enclose the inner drug core tablet. A PVA suture tab is inserted at this end of the device. The liquid monomer mixture and the silicone adhesive are cured by heating the assembly at 80° C. for 4 hours.

The examples and illustrated embodiments demonstrate some of the sustained release drug delivery device designs for the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

What is claimed:

1. A drug delivery device comprising;
a holder made of a material impermeable to passage of a pharmaceutically active agent, and including at least one opening for passage of the active agent therethrough;
a drug core contained in the holder, and including a pharmaceutically active agent; and
a membrane made of a material permeable to passage of the active agent, the membrane is contained in the holder and disposed between the drug core and the at least one opening in the holder; the improvement comprising:
the membrane is made of a crosslinked polyurethane-siloxane-containing copolymer.

2. The device of claim 1, further comprising a suture tab attached to the holder.

3. The device of claim 1 wherein the polyurethane-siloxane-containing copolymer is crosslinked with at least one hydrophilic monomer.

4. The device of claim 3 wherein the hydrophilic monomer(s) is present to total weight % of the comonomer mix from about 0.5% to 80%.

5. The device of claim 3 wherein the hydrophilic monomer(s) is present to total weight % of the comonomer mix from about 10% to 30%.

6. The device of claim 3 wherein the hydrophilic monomer is selected from the group consisting of ethylenically unsaturated lactam-containing monomers such as N-vinyl pyrrolidinone; methacrylic and acrylic acids; (meth)acrylic substituted alcohols, such as 2-hydroxyethylmethacrylate (HEMA) and 2-hydroxyethylacrylate; and (meth)acrylamides, such as methacrylamide and N,N-dimethyl acrylamide (DMA); vinyl carbonate or vinyl carbamate monomers; oxazolinone monomers; glycerol methacrylate, polyethyleneglycol monomethacrylate and mixtures thereof.

7. The device of claim 3 wherein the hydrophilic monomer is N,N-dimethyl acrylamide.

8. The device of claim 7 wherein the N,N-dimethyl acrylamide is present to total weight % of the comonomer mix from about 0.5% to 80%.

9. The device of claim 8 wherein the N,N-dimethyl acrylamide is present to total weight % of the comonomer mix from about 10% to 30%.

10. The device of claim 1 wherein the pharmaceutically active agent is a hydrophobic pharmaceutically active agent.

11. The device of claim 10 wherein the hydrophobic pharmaceutically active agent is selected from the group consisting of fluocinolone acetonide, triamcinolone acetonide, loteprednol etabonate, dexamethasone, acetazolamide, indomethacin, amphotericin B, paclitaxel, abecarnil, cyclosporines and mixtures thereof.

12. A drug delivery device comprising a holder made of a material impermeable to passage of a pharmaceutically active agent, and including at least one opening for passage of the active agent therethrough; a drug core contained in the holder, and a preformed membrane made of a polyurethane-siloxane-copolymer crosslinked with N,N-dimethyl acrylamide.

13. The device of claim 12 wherein the drug core contains fluocinolone acetonide.

14. The device of claim 12 wherein the N,N-dimethyl acrylamide is present to total weight % of the comonomer mix from about 10% to 30%.

15. The device of claim 14 further comprising a suture tab.

16. A method of delivering a pharmaceutically active agent comprising:
administering to a patient in need thereof the device of claim 1.

17. The method of claim 16 wherein the device is the device of claim 14.

18. The method of claim 16 wherein the device is the device of claim 15.

19. The device of claim 1, wherein the drug core is coated with a polyurethane-siloxane-copolymer crosslinked with from about 10% to about 30% N,N-dimethyl acrylamide.

20. The device of claim 14, wherein the drug core is coated with a polyurethane-siloxane-copolymer crosslinked with from about 10% to about 30% N,N-dimethyl acrylamide.

21. The device of claim 15, wherein the drug core is coated with a polyurethane-siloxane-copolymer crosslinked with from about 10% to about 30% N,N-dimethyl acrylamide.
